(19) United States
(12) Patent Application Publication
(10) Pub. No.: US 2006/0147411 A1
(43) Pub. Date: Jul. 6, 2006

(54) Novel compounds for the treatment of sickle cell disease

(75) Inventors: Ronald D. Guiles, Columbia, MD (US); Magdalena Guiles, Columbia, MD (US)

Correspondence Address: UNIVERSITY OF MARYLAND, BALTIMORE;TEC-COM/ORD, 515 W. LOMBARD STREET, BALTIMORE, MD, 21201, US

(73) Assignee: University of Maryland, Baltimore, Baltimore, MD (US)

(21) Appl. No.: 10/542,313

(22) PCT Filed: Jan. 30, 2004
(86) PCT No.: PCT/US04/02543

§ 371 Date: Jul. 15, 2005

Related U.S. Application Data
(60) Provisional application No. 60/443,783, filed on Jan. 30, 2003.

(51) Int. Cl. C08G 63/91; A61K 31/795

(52) U.S. Cl. 424/78.27; 525/54.2

(57) ABSTRACT

Compounds have been designed to inhibit the action of cytochrome b5 in the physiological re-reduction of auto-oxidized hemoglobin (methemoglobin), for the purpose of increasing methemoglobin levels in the blood of patients as a treatment for sickle cell disease. Administration of the compounds mimics congenital deficiencies in cytochrome b5, in which methemoglobin levels rise as high as 50% of total hemoglobin and derivatives in the blood, without adverse clinical manifestations. Methemoglobin inhibits red cell sickling and high levels of methemoglobin in the blood induced by the compounds of this invention prevent the symptoms of sickle cell disease.

This application claims the benefit of U.S. Provisional Application No. 60/443,783, filed Jan. 30, 2003, incorporated herein by reference.

### BACKGROUND OF THE INVENTION

1. Field of the Invention

This invention relates to compounds that bind to and inhibit the activity of cytochrome b₅ in the physiological re-reduction of auto-oxidized hemoglobin (methemoglobin), e.g., compounds that interfere with the binding of hemoglobin and/or methemoglobin to cytochrome b₅. The invention further relates to pharmaceutical compositions comprising these compounds, and methods of using these pharmaceutical compositions to increase methemoglobin levels in the blood as a treatment for sickle cell disease.

2. Description of the Related Art

In the United States the occurrence of sickle cell disease, also known as sickle cell anemia, is relatively low, afflicting only about 70,000 Americans (Jones, C. P. 1995, Pharmacy Today, 1, 1-2), predominantly of African descent. Worldwide, however, sickle cell disease afflicts many millions of individuals (Bunn, H. F.; Forget, B. G. Hemoglobin: Molecular, Genetic and Clinical Aspects; W. B. Saunders Company: Philadelphia, 1986. 502-564). Despite the fact that the molecular mechanism of hemoglobin sickling is well understood (Eaton, W. A.; Hofrichter, J. 1990, Adv. Protein Chem., 40, 63-279) and the role of sickling in the pathology of the disease is clear, a rationally based drug therapy has not heretofor been available to patients despite many attempts to develop such a therapy over the last few decades (Orringer, E. P.; Casella, J. P.; Ataga, K. I.; Koshy, M.; Adams-Graves, P.; Luchtman-Jones. L.; Wun, T.; Watanabe, M.; Shafer, F.; Kutlar, A.; Abboud, M.; Steinberg, M.; Adler, B.; Swerdlow, P.; Terregino, C.; Saccente, S.; Files, B.; Ballas, S.; Brown, R.; Wojtowicz-Praga, S.; Grindel, J. M. 2001, JAMA, 286(17):2099; Abraham, D. J.; Perutz, M. F.; Phillips, S. B. 1983, Proc. Natl. Acad. Sci. USA, 80, 324-328; Klotz, I. M.; Haney, D. N.; King, L. C. 1981 Science,213, 724-731). Although agents that modify hemoglobin allosterism have been identified through site-directed drug design (Abraham, D. J.; Wireko, F. C.; Randad, R. S.; Poyart, C.; Kister, J.; Bohn, B.; Liard, J. F.; Kunert, M. P. 1992, Biochemistry, 31, 9141-9149), to date, attempts at hemoglobin-directed antisickling agents have been unsuccessful.

The clinical manifestations of sickle cell disease are highly variable (Bunn, H. F.; Forget, B. G. Hemoglobin: Molecular, Genetic and Clinical Aspects; W. B. Saunders Company: Philadelphia, 1986. 502-564; Serjeant, G. R. 2001, Br. J. Haematol., 112, 3-18). In young children, the major concern is the incidence of stroke. Chronic hemolytic anemia, impairment of growth and higher susceptibility to infection are common systemic manifestations. Vaso-occlusive crises are the origin of the most severe symptoms, including stroke and cardiac involvement in cases of “chest syndrome”, The incidence of three or more vaso-occlusive crises per year is highly correlated with mortality. With current treatment, life expectancy for sickle cell patients is 40 to 50 years.

Until recently, the only approach to the treatment of sickle cell disease was fluids and analgesics, such as morphine, administered upon the occurrence of vaso-occlusive crises.

While transfusion therapy is commonly employed in pediatric cases where the risk of stroke is high, there are serious potential problems with long-term transfusion therapy (Serjeant, G. R. 2001, Br. J. Haematol., 112, 3-18). The risk of stroke is predicted by transcranial doppler measurements of blood flow in the brain. Although transfusion therapy is effective in reducing vaso-occlusive crises, such as stroke, there are several drawbacks. Iron overload is a common side effect, and iron chelation therapy employing desferoxamine is a common adjuvant therapy. Long-term transfusion therapy also carries the risk of alloimmunogenic reactions. There is also a risk of disease transmission that has been minimized with recent advances in diagnostic procedures.

In 1995, hydroxyurea (Jones, C. P. 1995 Siclde Cell Therapy so Effective, Trials end early, Pharmacy Today, 1, 1-2; Charache, S.; Terrin, M. L.; Moore, R. D.; Dover, G. J.; Barton, F. B.; Eckert, S. V.; McMahon, R. P.; Bonds, D. R. 1995, N. Engl. J. Med., 332, 317-1322; Rodgers, G. P. 1997 Semin. Hematol., 34, 2-7.) became available for the treatment of sickle cell disease. Because it was already used in the treatment of certain leukemias, it was rapidly approved for clinical testing and passed through clinical trials faster than any other drug in recent times. In an extensive study involving nearly 300 sickle cell patients, the occurrence of vaso-occlusive crises was reduced to roughly 50% of that observed in the patients involved (Charache, S.; Terrin, M. L.; Moore, R. D.; Dover, G. J.; Barton, F. B.; Eckert, S. V.; McMahon, R. P.; Bonds, D. R. 1995, N. Engl. J. Med., 332, 317-1322.). It is believed that hydroxyurea works by inducing expression of fetal hemoglobin, however, there are a number of controversies concerning the exact mechanism of action, given that benefits appear to begin before the development of significant levels of fetal hemoglobin (Bunn, H. F. 1999, Blood, 93, 1787-1789). Despite the advantages in the use of hydroxyurea, it is not without significant side effects. Hydroxyurea is myelosuppressive and thus patients must be monitored carefully (Rodgers, G. P. 1997 Semin. Hematol., 34, 2-7). Hydroxyurea causes chromosomal fragmentation and is teratogenic and mutagenic but does not appear to be carcinogenic. Because of the mutagenicity and the potential carcinogenicity in the long-term, it is not approved for use in children. Rather, it is approved for use in patients who suffer more than three vaso-occlusive crises a year, a clinical pattern strongly correlated with mortality (Castro, O. 1999, Br. J. Haematol., 107, 2-11).

Relatively recently, bone marrow transplantation has been found to be an effective cure for sickle cell disease (Serjeant, G. R. 2001, Br. J. Haematol., 112, 3-18). This treatment was first discovered when a leukemia patient was given a bone marrow transplant and serendipitously was also cured of his sickle cell disease. Several hundred bone marrow transplants have been performed specifically for the purpose of treating sickle cell disease. This approach is only available to about 18% of sickle cell patients because of the requirement of an HLA matched sibling donor. The procedure is costly and carries significant risks. Mortality because of immune responses ranges from 10% to 15% and subsequent alloimmune responses can be problematic. Thus, although bone marrow transplantation is a very promising cure for the genetic disorder, it has significant limitations that prevent widespread use.

It is known that methemoglobin, oxyhemoglobin, and carbonmonoxyhemoglobin, effectively inhibit sickling in patients with siclde cell disease (Franklin, I. M.; Rosemeyer, M. A.; Huehns, E. R. 1983, Br. J. Haematol., 54, 579-587). Furthermore, in individuals with congenital deficiencies in cytochrome b₅, methemoglobin levels rise as high as 50% of total hemoglobin and derivatives in the blood, without any adverse clinical manifestations other than mild cyanosis. Clinical trials performed in the 1960's demonstrated the efficacy of methemoglobin in the suppression of vaso-occlusive crises but were limited by the rapid re-reduction of methemoglobin by cytochrome b₅ and hence required massive quantities of compounds such as sodium nitrite, benzocaine or para-aminopropriophenone to maintain sufficient levels of methemoglobin (Beutler, E. 1961, J. Clin. Invest., 40, 56-68). Delivery of the appropriate quantities of these compounds was difficult and the prospect of good patient compliance with such a drug regimen was remote. Thus, sickle cell disease is a serious problem for which no effective solution is available, and a potentially useful approach to the treatment of the disease would be to increase the amount of methemoglobin in patients having siclde cell disease.

Cytochrome b₅ is the terminal electron donor to methemoglobin in the physiological re-reduction of auto-oxidized hemoglobin (Abe, K.; Sugita, Y. 1979, Eur. J. Biochein., 101, 423-428; Gerbaut, L. 1991 Clin. Chem., 37, 2117-2120). Hemoglobin auto-oxidizes at approximately 3% per day. The structures for hemoglobin and its derivatives have been previously determined (Perutz, M. F. 1989 TIBS, 14, 42-44; Bolton, W.; Cox, J. M.; Perutz, M. F. 1968, J Mol Biol, 33, 283-297) and as well as that of cytochrome b₅ (Mathews, S.; Czerwinski, E. W.; Argos, P. The X-Ray Crystallographic Structure of Calf Liver Cytochrome b₅; Dolphin, D., Ed.; Academic Press: New York, 1979; Vol. VII, pp 107-147). Complete NMR assignments for the rat cytochrome b₅ were determined for both equilibrium forms (Guiles, R. D.; Basus, V. J.; Kuntz, I. D.; Waskell, L. 1992, Biochemistry, 31, 11365-11375; Guiles, R. D.; Basus, V. J.; Sarma, S.; Malpure, S.; Fox, K. M.; Kuntz, I. D.; Waskell, L. 1993, Biochemistry, 32, 8329-8340). In addition, extensive characterization of the structural, dynamic, and electrochemical properties of rat cytochrome b₅ have been performed (Dangi, B.; Sarma, S.; Yan, C.; Banville, D. L.; DiGate, R. J.; Guiles, R. D. 1998, Biochemistry,37, 8289-830251-54; Dangi, B.; Blankman, J. I.; Miller, C. J.; Volkman, B. F.; Guiles, R. D. 1998, J. Phys. Chem B,102, 8201-8208; Sarma, S.; Banville, D.; DiGate, R. J.; Miller, C.; Guiles, R. D. 1997, Biochemistry,36, 5658-5668; Cheng, J.; Terrettaz, S.; Blankman, J. I.; Miller, C. J.; Dangi, B.; Guiles, R. D. 1997, Israel Journal of Chemistry,37, 259-266; Blankman, J. I.; Shahzad, N.; Dangi, B.; Miller, C. J.; Guiles, R. D. 2000, Biochemistry,39, 14799-14805). Furthermore, theoretical studies of the hemoglobin and cytochrome b₅ complex have been performed (Poulos, T. L.; Mauk, A. G. 1983, J. Biol. Chem., 258, 7369-7373).

### SUMMARY OF THE INVENTION

The present invention is directed to compounds that inhibit cytochrome b₅'s action in the re-reduction of methemoglobin to hemoglobin, which thereby leads to an increase in methemoglobin levels. Thus, the compounds of the present invention are useful in treating sickle cell disease.

According to a preferred embodiment, the present invention provides a compound of the formula R1-R2-R3, wherein: R1 comprises a moiety that binds to the hemoglobin binding site on cytochrome b₅ and competitively inhibits hemoglobin binding to cytochrome b₅; R3 comprises a moiety that binds to cytochrome b₅ at a site distinct from the site at which R1 binds to cytochrome b₅; and R2 comprises a moiety that links R1 and R3.

In one preferred embodiment of a compound of the present invention, R1 is a linear polyamine.

In another preferred embodiment of a compound of the present invention, R1 is a cyclic polyamine.

In another preferred embodiment of a compound of the present invention, R1 is a hexacyclen.

In another preferred embodiment of a compound of the present invention, R1 is a moiety that binds to cytochrome b₅ at one or more amino acids selected from the group consisting of H26, E43, E44, E48, A54, D60, H80 and A88.

In another preferred embodiment of a compound of the present invention, R3 is a moiety that binds to the ATP binding site on cytochrome b₅.

In another preferred embodiment of a compound of the present invention, R3 is ATP or an ATP analog.

In another preferred embodiment of a compound of the present invention, R3 is β-nicotinamide adenine dinucleotide.

In another preferred embodiment of a compound of the present invention, R3 is ATP; 1,N6-ethenoadenosine 5′ triphosphate; β-nicotinamide adenine dinucleotide; 1,N6-ethenoadenosine hydrochloride; nicotinamide-1,6-ethenoadenosine; or coenzyme A.

In another preferred embodiment of a compound of the present invention, R3 is a moiety that binds to cytochrome b₅ at one or more amino acids selected from the group consisting of I24, L25, H26 and H27.

In a particular preferred embodiment of a compound of the invention, R1 is hexacyclen and R3 is β-nicotinamide adenine dinucleotide.

In one preferred embodiment of a compound of the present invention, R2 is a flexible linker.

In another preferred embodiment of a compound of the present invention, R2 is a moiety that covalently crosslinks R1 and R3.

In another preferred embodiment of a compound of the present invention, R2 is a polyglycine moiety.

In another preferred embodiment of a compound of the present invention, R2 is polyethylene glycol (PEG); polystyrene-PEG; [2-(2-aminoethoxy)ethoxy] acetic acid; allyloxycarbonyl-[2-(2-aminoethoxy)ethoxy] acetic acid; fluorenyl-methoxycarbonyl-[2-(2-aminoethoxy)ethoxy] acetic acid; ter-butyloxycarbonyl-[2-(2-aminoethoxy)ethoxy] acetic acid; benzyloxycarbonyl-[2-(2-aminoethoxy)ethoxy] acetic acid; or BMPS (N-(β-maleimido-propyloxy)succinimide).

In another preferred embodiment of a compound of the present invention, R2 is a straight chain or branched chain hydrocarbon.

In a preferred embodiment of a compound of the present invention, said compound binds to cytochrome b₅ and inhibits the activity of cytochrome b₅ in the reduction of methemoglobin to hemoglobin.

According to another preferred embodiment, the present invention provides a pharmaceutical composition comprising a compound of the present invention or a pharmaceutically acceptable salt thereof.

According to another preferred embodiment, the present invention provides a method of reducing the incidence of red blood cell sickling in a patient with sickle cell disease, comprising administering an effective amount of a compound of the present invention to the patient.

According to another preferred embodiment, the present invention provides a method of raising the level of methemoglobin in blood, comprising adding an effective amount of a compound of the invention to the blood. In one embodiment of this method, the compound is added to the blood ex vivo.

According to another preferred embodiment, the present invention provides a method of raising the level of methemoglobin in the blood of a patient, comprising administering an effective amount of a compound of the present invention to the patient.

### BRIEF DESCRIPTION OF THE DRAWINGS

FIGS. 1A and 1B illustrate sections of contour plots of overlays of ¹H-¹⁵N HSQC spectra of cytochrome b₅ by itself and in complex with human methemoglobin. In FIG. 1A, the concentration of cytochrome b₅ is 1 mM and the concentration of methemoglobin is 0.50 mM. In FIG. 1B, the concentration of methemoglobin is 0.25 mM.

FIG. 2 illustrates the heteronuclear correlation spectra (HSQC spectra) of a 2 mM solution of cytochrome b₅ by itself (black contours) and that of a solution containing 2 mM cytochrome b₅ and 4 mM hexacyclen (gray contours).

FIG. 3 shows modification of hexacyclen to enable attachment of an R2 linker for use in crosslinking to the R3 moiety.

FIG. 4 shows the thiolation of ADP that can then be linked to the derivatized polyamines.

FIG. 5 shows linking of derivatized spermine to derivatized ADP.

FIG. 6 shows attachment of the flexible spacer and covalent attachment of the two derivatized groups.

FIG. 7 shows an HSQC overlay of a sample containing cytochrome b₅ and ATP (2 mM) and a sample of cytochrome b₅ alone.

FIG. 8 shows a set of traces of the optical absorbance changes occurring at 577 nm for cytochrome b₅ and methemoglobin at 5 μM concentrations. Various concentrations of buffer and of hexacyclen were examined. In trace A) the buffer concentration is 10 mM phosphate at pH 7.0. In trace B) the buffer concentration is 1 mM phosphate at pH 7.0. In trace C) the buffer concentration is 1.0 mM phosphate pH 7.0 and the concentration of hexacyclen is 100 μM. In trace D and E) the concentration of phosphate is 1 mM pH 7.0 and the concentration of hexacyclen is 1 mM.

### DETAILED DESCRIPTION OF THE INVENTION

The present invention provides compounds having the structure R1-R2-R3 that bind to cytochrome b₅ and inhibit the activity of cytochrome b₅ in the reduction of methemoglobin to hemoglobin. Without wishing to be bound to any particular mechanism, it is proposed that these compounds prevent the binding of hemoglobin and/or methemoglobin to cytochrome b₅ by binding with high affinity to the methemoglobin/hemoglobin binding site on cytochrome b₅ and preventing the electron transfer between methemoglobin/hemoglobin and cytochrome b₅. By binding to cytochrome b₅ and preventing reduction of autoxidized hemoglobin, these compounds raise the level of methemoglobin in red blood cells and reduce the incidence of cell sickling. Thus, these compounds are useful for the treatment of sickle cell disease.

In one embodiment, the invention relates to a compound that comprises three parts, designated R1, R2 and R3 as described below. R1 and R3 bind to specific sites on the surface of cytochrome b₅ as defined by shifts in ¹H-¹⁵N heteronuclear correlation spectrum peaks defined below (Heteronuclear Single Quantum Coherence (HSQC) mapping: Mori, S.; Abeygunawardana, C.; Johnson, M. 0. N.; van Zijl, P. C. M. 1995, Journal of Magnetic Resonance, Series B, 108, 94-98). R2 is a linker which covalently links R1 and R3.

R1 is a moiety that binds to specific sites on cytochrome b₅ in a way that mimics hemoglobin binding to cytochrome b₅, except preferably with higher affinity than hemoglobin binding. Thus, R1 competitively inhibit hemoglobin binding to cytochrome b₅. Furthermore, the high affinity of R1 to the hemoglobin binding site of cytochrome b₅ inhibits electron transfer from cytochrome b₅ to methemoglobin.

One can use an optical assay of electron transfer rate to identify such moieties (see examples below). It is believed that moieties that interfere with electron transfer between cytochrome b₅ and methemoglobin also bind to one or more of the following cytochrome b₅ residues: H26, E43, E44, E48, A54, D60, H80 and A88, as shown by shifts in HSQC perturbation mapping (see examples below). (Shuker, S. B.; Hajduk, P. J.; Meadows, R. P.; Fesik, S. W. 1996, Science, 274, 1531-4).

In a preferred embodiment, R1 is 1,4,7,10,13,16-hexaazacylooctadecane (hexacyclen), the structure of which is as follows:

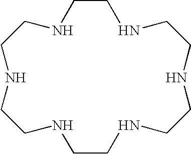

In other preferred embodiments, R1 is a derivative of hexacyclen, such as that described in Example 4.

R2 is a linker between R1 and R3. Although a number of linkers, flexible and non-flexible, are known in the art field, it is preferable that the linker, R2, be flexible. In one embodiment, R2 is a polyglycine moiety containing between 1 and 3 glycines. In another embodiment, R2 is polyethylene glycol (PEG), or a PEG-like moiety such as, but not limited to, polystyrene-PEG, [2-(2-aminoethoxy)ethoxy] acetic acid, allyloxycarbonyl-[2-(2-aminoethoxy)ethoxy] acetic acid, fluorenyl-methoxycarbonyl-[2-(2-aminoethoxy)ethoxy] acetic acid, ter-butyloxycarbonyl-[2-(2-aminoethoxy)ethoxy] acetic acid, or benzyloxycarbonyl-[2-(2-aminoethoxy)ethoxy] acetic acid. In still additional embodiments, R2 is a straight chain or branched chain of carbon and hydrogen where the number of carbon atoms is 3, 4, 5, 6, 7, 8, 9, 10, 11, 12, 13, 14, 15, 20, 25, 30, or more. An appropriate-length linker connects R1 and R3 when they are both bound to their respective binding sites on cytochrome b₅.

R3 is a moiety that binds to a site on cytochrome b₅ distinct from the binding site of R1. Preferably, R3 binds to specific sites on cytochrome b₅ in a way that mimics ATP binding to cytochrome b₅. This additional and distinct binding of R3 to cytochrome b₅ increases the overall affinity of a compound of the present invention for cytochrome b₅. Thus, even at the relatively high salt concentration of blood, compounds of the present invention bind with high affinity to cytochrome b₅.

It is preferable that R3 binds to cytochrome b₅ and induces shifts in heteronuclear correlation peaks corresponding to one or more of following residues on cytochrome b₅: I24, L25, H26, and H27. In a preferred embodiment of the invention, R3 is ATP (adenosine 5′-triphosphate); 1,N6-ethenoadenosine 5′ triphosphate; β-nicotinamide adenine dinucleotide; 1,N6-ethenoadenosine hydrochloride; nicotinamide-1,6-ethenoadenosine; or coenzyme A. In another preferred embodiment of the invention, R3 is any ATP analog, many of which exist and are well known in the art-field. ATP binds to cytochrome b₅ with an affinity of 180 μM. Below are the chemical structures of some R3 moieties according to preferred embodiments of the invention, all of which are commercially available: 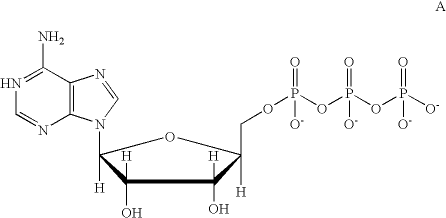
Adenosine 5′-triphosphate (ATP); 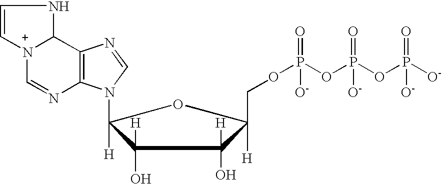
1,N6-Ethenoadenosine 5′ triphosphate; 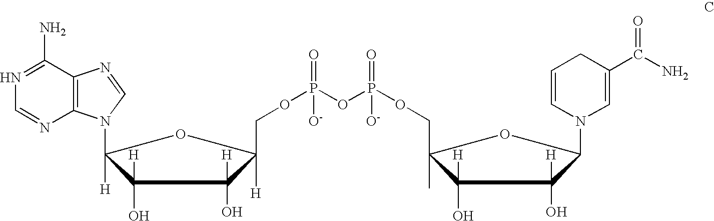
β-Nicotinamide adenine dinucleotide; 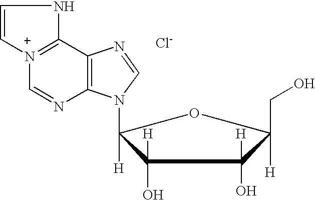
1,N6-ethenoadenosine hydrochloride; 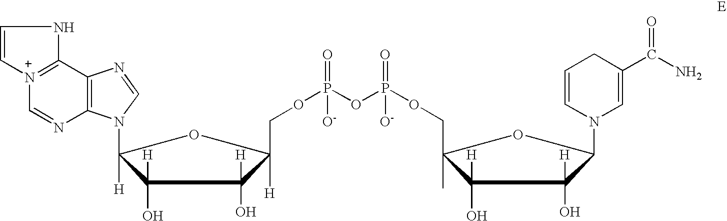
Nicotinamide-1,6-ethenoadenosine; 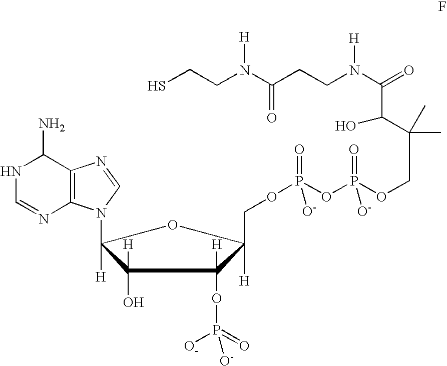
Coenzyme A.

The present invention further provides a method of reducing the incidence of red blood cell sickling in a patient with sickle cell disease and in need of treatment thereof, comprising administering an effective amount of a compound according to the present invention to the patient.

This invention also provides a method for preventing the reduction of methemoglobin to hemoglobin such that methemoglobin accumulates in the blood, comprising administering an effective amount of a compound according to the present invention. Such an accumulation of methemoglobin is useful for the prevention of sickling events in patients having sickle cell disease. Thus, the present invention provides a method of raising the level of methemoglobin in the blood of a patient, comprising administering an effective amount of a compound according to the present invention to the patient.

The invention also provides a method of raising the level of methemoglobin in blood, comprising adding an effective amount of a compound of the invention to the blood. For example, the compound can added to the blood ex vivo. This blood can then be used to transfuse a patient having sickle cell disease.

Cytochrome b₅ plays an important role in reducing methemoglobin levels, as demonstrated experimentally and indicated in individuals with congenital deficiencies in cytochrome b₅, who have abnormally high levels of methemoglobin in their blood. Compounds of the invention can inhibit the activity of cytochrome b₅ by, e.g., blocking the binding of methemoglobin to cytochrome b₅. Compounds of the invention can also achieve the inhibition of cytochrome b₅ activity by, e.g., blocking electron transfer to methemoglobin. Compounds of the invention that are comprised of two moieties such that each moiety binds to different sites on cytochrome b₅ have an affinity for cytochrome b₅ that is greater than the affinity of either moiety for its individual site. The compounds of the invention are thus highly effective at inhibiting cytochrome b₅ activity and raising levels of methemoglobin in the blood.

The inventive compounds exhibit therapeutic activity in raising levels of methemoglobin in the blood, and are effective in treating sickle cell disease by reducing the amount of cell sickling. In accordance with a preferred embodiment, the present invention includes methods of treating patients suffering from sickle cell disease.

The present invention further provides pharmaceutical compositions comprising a compound according to the present invention and a pharmaceutically acceptable carrier; a method of inhibiting the activity of cytochrome b₅ in red blood cells by administering a pharmaceutical composition of the invention to a patient; a method of increasing the levels of methemoglobin in red blood cells by administering a pharmaceutical composition of the invention to a patient; and a method of treating sickle cell disease in a patient by administering a pharmaceutical composition of the invention to the patient.

The present invention also relates to useful forms of the compounds as disclosed herein, such as pharmaceutically acceptable salts and prodrugs of all the compounds. The compounds of the invention can be administered alone or as an active ingredient of a formulation. Thus, the present invention also includes pharmaceutical compositions of compounds of the invention containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Isol, editor), 1553-1593 (current edition).

In view of the high degree of selective inhibition of cytochrome b₅ activity, the compounds of the present invention can be administered to a patient requiring inhibition of cytochrome b₅ activity. Administration may be accomplished according to patient's needs, for example, by intravenous injection. Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time-release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

The compounds can be administered as the sole active agent or in combination with other pharmaceutical agents, such as other agents that raise levels of hemoglobin variants in the red blood cells in order to prevent cell sickling in patients with sickle cell disease.

The dosages of the compounds of the present invention depend upon a variety of factors including the severity of the symptoms, the age, sex and physical condition of the patient, the route of administration, the frequency of the dosage interval, the particular compound utilized, the efficacy, toxicology profile, pharmacokinetic profile of the compound, and the presence of any deleterious side-effects, among other considerations.

By “effective dose” or “therapeutically effective dose” or “effective amount” is meant herein, in reference to the treatment of sickle cell disease, an amount sufficient to bring about one or more of the following results: increase the level of methemoglobin in the blood above about 3%; reduce the level of pain related to sickle cell disease; or reduce the incidence of sickle cell crises. The compounds of the invention can be administered at dosage levels and in a manner customary for ticlopidine hydrochloride, or other drugs used to treat sickle cell disease. For example, ticlopidine hydrochloride is administered at 250 mg bi-daily (see Physicians' Desk Reference, the relevant portion of which incorporated herein by reference). However, the concentration of cytochrome b₅ in blood is 5000 times lower than the concentration of hemoglobin (0.2 μM compared to 1 mM). Therefore, a compound of the invention targeted to cytochrome b₅ could potentially be administered at a dose of up to 5000 times lower than the dose of a sickle cell drug that is targeted to hemoglobin, e.g. ticlopidine hydrochloride. By this extrapolation, a compound of the invention could be administered at a dose of only 50 μg twice daily.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

### EXAMPLES

### Example 1

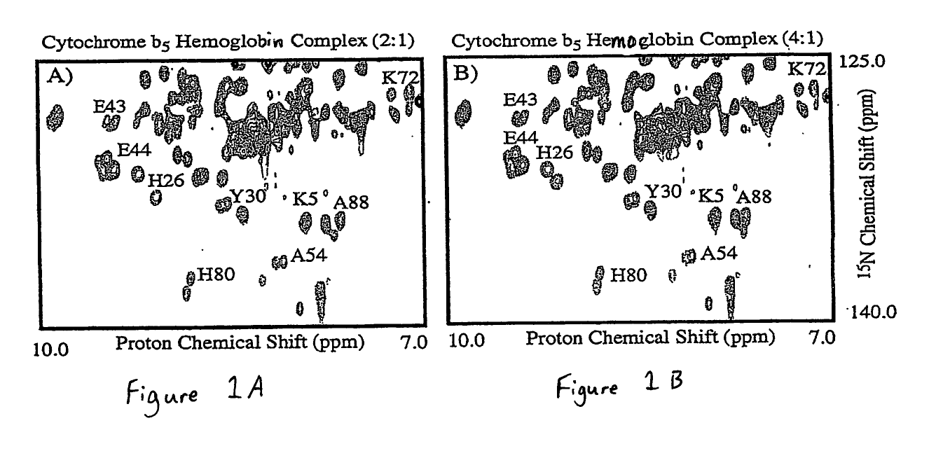

The interaction of cytochrome b₅ with hemoglobin is explored using HSQC perturbation mapping. FIG. 1 contains sections of contour plots of overlays of ¹H-¹⁵N HSQC spectra of cytochrome b₅ by itself and in complex with human methemoglobin. Significant shifts in the positions of a number of residues of cytochrome b₅ are observed. In FIG. 1A, the concentration of cytochrome b₅ is 1 mM and the concentration of methemoglobin is 0.50 mM. The black contours are of a heteronuclear correlation spectrum of cytochrome b₅ by itself while the gray contours are of a sample containing both cytochrome b₅ and methemoglobin. In FIG. 1B, the concentration of methemoglobin is 0.25 mM. The pH in all cases was 6.4 and the temperature was 25° C. A number of residues that shift significantly on complex formation are labeled (e.g. most notably H26, E43, E44, A54, H80 and A88). A number of peaks that do not shift significantly on complex formation are also labeled (i.e. K5 and Y30). Heteronuclear correlation spectra were recorded using the fast HSQC sequence (Mori, S.; Abeygunawardana, C.; Johnson, M. O. N.; van Zijl, P. C. M. 1995, J. Mag. Reson. B, 108, 94-98). The shifts in heteronuclear correlation peaks observed on complex formation are consistent at least in part with the theoretical model of the complex between cytochrome b₅ and methemoglobin. The shifts in peaks associated with residues E43, E44 and probably A54 via a relayed effect in helix V of cytochrome b₅ are consistent with the theoretical model. Compounds which interact with cytochrome b₅ at one or more of the following amino acids of cytochrome b₅ are proposed to interfere with or prevent the binding of methemoglobin to cytochrome b₅, the amino acids being H26, E43, E44, A54, H80 and A88.

### Example 2

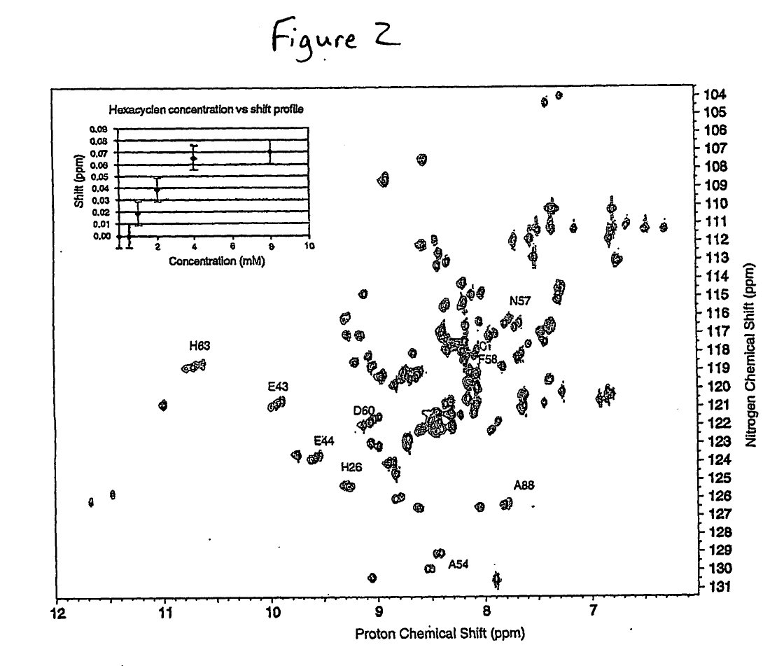

Hexacyclen (1,4,7,10,13,16-hexaazacyclooctadecane)(Richman, J. E.; Atkins, T. J. 1974, J. Am. Chem. Soc., 96, 2268-2269) binds to cytochrome b₅ such the HSQC spectra of cytochrome b₅-hexacyclen is similar to the HSQC spectra of cytochrome b₅-hemoglobin. The concentration dependence of hexacyclen-induced heteronuclear correlation peak shifts indicates a dissociation constant of roughly 2 mM. FIG. 2 illustrates the heteronuclear correlation spectra (HSQC spectra) of a 2 mM solution of cytochrome b₅ by itself (black contours) and that of a solution containing 2 mM cytochrome b₅ and 4 mM hexacyclen (gray contours). The inset in the upper left hand corner of the figure contains a plot of the hexacyclen dependence of the shifts in the peak to peak separation of aspartate 60 (D60) at concentrations of hexacylen ranging from 0.5 to 8 mM. The inset at the upper right is a model for the interaction of hexacyclen based on the shifts observed in the HSQC perturbation study. Solutions were buffered to a pH of 7.0 with 1 mM phosphate buffer and the spectra were recorded at 40° C. The insert in the upper left hand corner of the figure contains a plot of the hexacyclen dependence of the shifts in the peak to peak separation of aspartate 60 (D60) at concentrations of hexacylen ranging from 0.5 to 8 mM. The insert at the upper right is a model for the interaction of hexacyclen based on the shifts observed in the HSQC perturbation study.

### Example 3

Work has been performed on cytochrome b₅ in an attempt to find compounds which inhibit or increase cytochrome b₅'s properties, such as the reduction potential and binding affinity to cytochrome b₅'s electron transfer partners by relayed effects (Rivera, M.; Wells, M. A.; Walker, F. A. 1994, Biochemistry, 33, 2161-2170; Vergeres, G.; Waskell, L. 1995, Biochemie, 77, 604-620; Reid, L. S.; Gray, H. B.; Dalvit, C.; Wright, P. E.; Saltman, P. 1987, Biochem., 26, 7102-7107). ATP binds to cytochrome b₅ with an affinity of 180 μM (Reid, L. S.; Gray, H. B.; Dalvit, C.; Wright, P. E.; Saltman, P. 1987, Biochem., 26, 7102-7107). ATP binds to a hydrophobic domain between the four helix bundle that binds the heme and the β-turn in the β-sheet region on cytochrome b₅, this binding site being distinct from the binding site of hexacyclen.

### Example 4

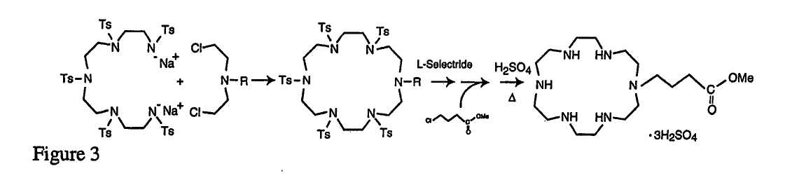

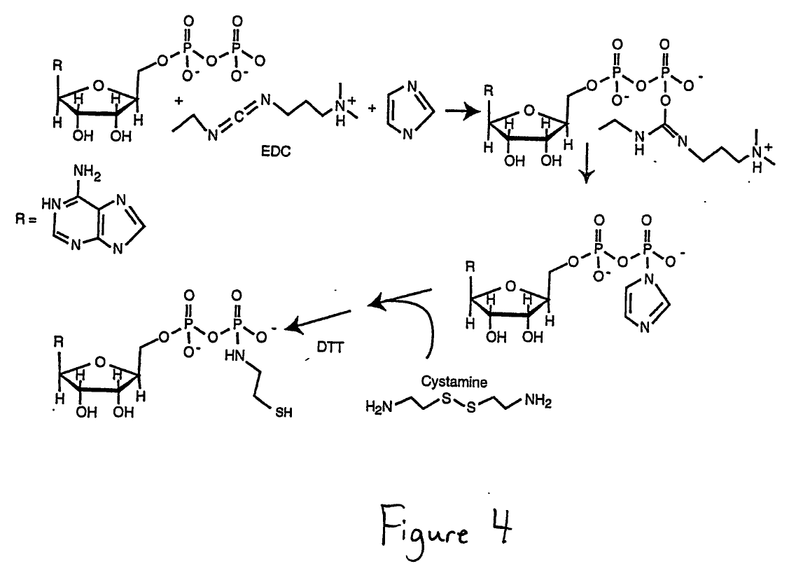

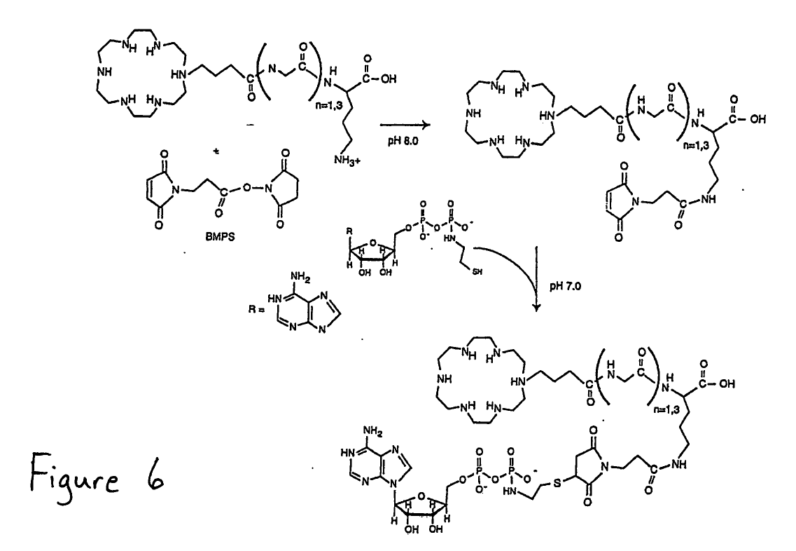

Schemes for the derivatization of hexacyclen (R1) have been developed using minor modifications of the Richman-Atkins synthesis(Richman, J. E.; Atkins, T. J. 1974, J. Am. Chem. Soc., 96, 2268-2269). The R group in the scheme shown in FIG. 3 was a tosyl group in the original synthesis, but has been replaced with a carbamyl group in this modification, which can be selectively removed using L-selectride (Coop, A.; Rice, K. C. 1998, Tet. Lett., 39, 8933-8934), lithium tri-sec-butylborohydride (Aldrich), under mild conditions which will not remove the Ts group. FIG. 3 shows a modified Richman-Atkins synthesis of hexacyclen to enable attachment of a derivatizable R group for use in crosslinking to the R3 moeity. A scheme utilizing this modified hexacyclen in the preparation of a polypeptide that is used to link the thiolated ADP shown in FIG. 4 using a bifunctional crosslinking agent is shown in FIG. 6.

### Example 5

In this scheme to link hexacyclen (R1) with an ADP derivative (R3), described by Hermanson (Hermanson, G. T. Bioconjugate Techniques; Academic Press, 1995.785 pp649-655), a water soluble carbodiamide is used to activate the terminal phosphate which can then be thiolated with β-mercaptoethylamine via cystamine following reductive cleavage with Cleland's reagent as shown in FIG. 4. As shown in FIG. 4, a water soluble carbodiamide is used to activate the terminal phosphate that can then be thiolated with β-mercaptoethylamine via cystamine following reductive cleavage with Cleland's reagent.

### Example 6

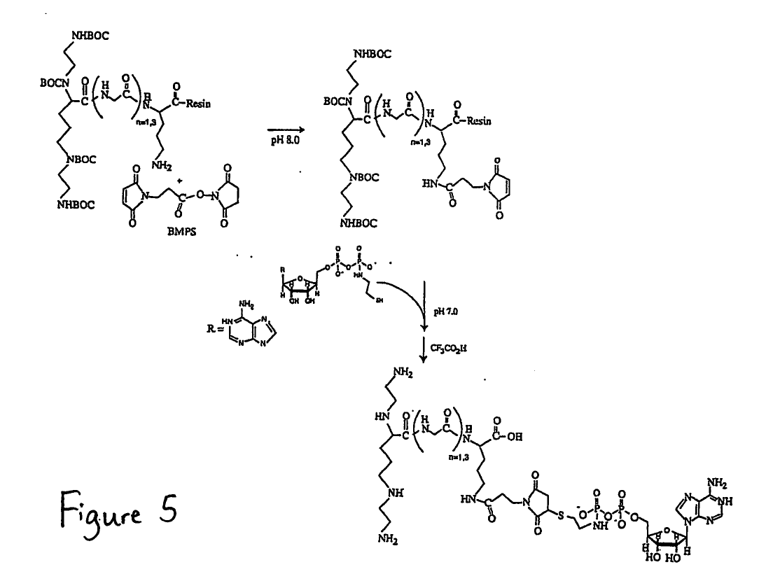

In this scheme to link hexacyclen (R1) with an ADP derivative (R3), a polypeptide containing a C-terminal lysine and N-termination with the derivatized spermine is prepared using standard solid phase methods (Grant, G. A. Synthetic Peptides: A User's Guide; W. H. Freeman and Company: New York, 1992, 382). This polypeptide is then crosslinked to the derivatized ADP shown in FIG. 3 using the bifunctional crosslinker BMPS (N-(β-maleimido-propyloxy)succinimide) (McKenzie, J. A.; Raison, R. I.; Rivett, E. E. 1988, J. Protein Chem., 7, 581-592) as shown in FIG. 5.

### Example 7

A similar scheme for linking a polypeptide with an N-terminal hexacylen derivative to the thiolated ADP is shown in FIG. 6. In this scheme a polypeptide with a C-terminal lysine and a variable number of glycines is prepared using standard solid phase synthesis techniques. The N-terminal group here is the derivatized hexacyclen.

### Example 8

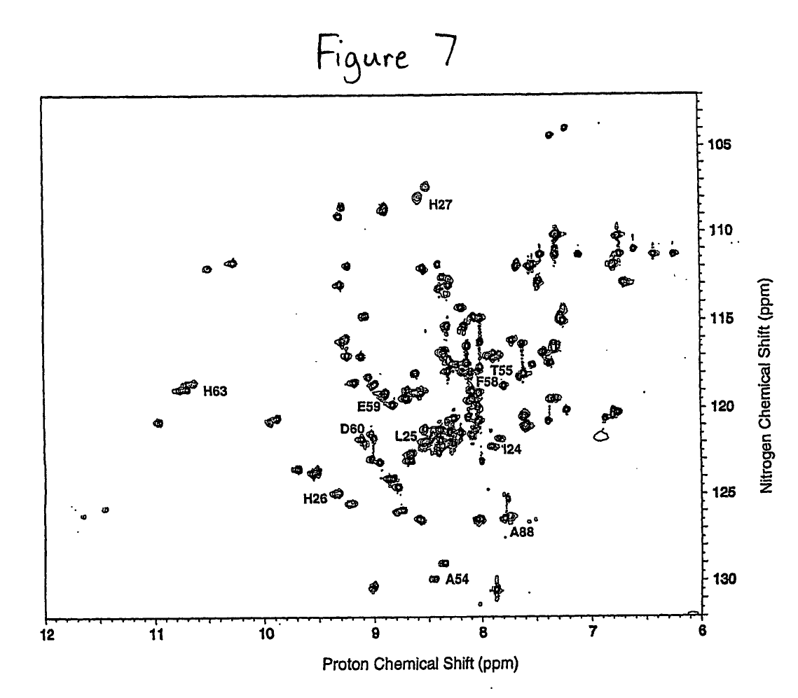

The binding of the R1 and R3 moieties to cytochrome b₅ is characterized using HSQC perturbation mapping experiments. FIG. 2 contains an overlay of heteronuclear correlation spectra (HSQC spectra) of a 2 mM solution of cytochrome b₅ by itself (black contours) and that of a solution containing 2 mM cytochrome b₅ and 4 mM hexacyclen (gray contours), illustrating shifts due to the binding of hexacyclen to ¹⁵N-labeled cytochrome b₅. FIG. 2 can be compared with FIG. 7, which contains an HSQC overlay of a control on that of a sample containing cytochrome b₅ and ATP. Solutions were buffered to a pH of 7.0 with 1 mM phosphate buffer and the spectra were recorded at 40° C. FIG. 7 contains an HSQC overlay of a control on that of a sample containing cytochrome b₅ and ATP at 2 mM concentration. Although there is some overlap in peaks affected by the binding of ATP with that seen with the binding of hexacyclen, some of these effects are probably relayed.

### Example 9

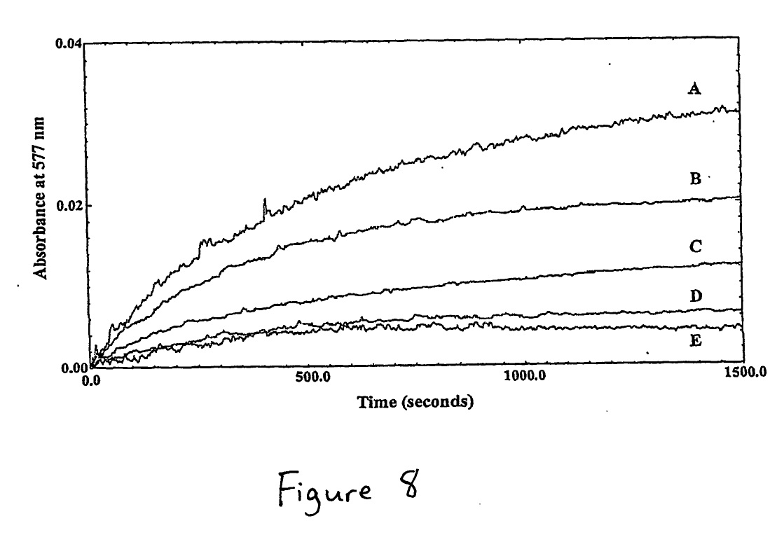

In addition to the. site interaction studies using NMR, functional assays of inhibition of electron transfer have been performed using manual mixing experiments, similar to those described by Sugita (Abe, K.; Sugita, Y. 1979, Eur. J. Biochem. 101, 423-428). The electron transfer reactions were monitored by observing absorbance changes at 577 nm similar to experiments performed by McLendon's group (Qiao, T.; Simmons, J.; Horn, D. A.; Chandler, R.; McLendon, G. 1993, 97, 13089-13091). FIG. 8 contains a set of traces of the optical absorbance changes occurring at 577 nm for cytochrome b₅ and methemoglobin at 5 μM concentrations with 1 mM phosphate buffer at pH 7.0. Various concentrations of hexacyclen were examined ranging from 100 μM to 1 mM. The concentration of phosphate buffer was also examined in order to assess the effect of ionic strength on the rate of the reaction. In all cases the concentration of cytochrome b₅ and methemoglobin is 5 μM and the temperature was maintained at 37° C. For trace A, the buffer concentration is 10 mM phosphate at pH 7.0. For trace B, the buffer concentration is 1 mM phosphate at pH 7.0. For trace C, the buffer concentration is 1.0 mM phosphate, pH 7.0 and the concentration of hexacyclen is 100 mM. For trace D and trace E, the concentration of phosphate is 1 mM, pH 7.0 and the concentration of hexacyclen is 1 mM.

While the disclosure above describes the invention in detail and with reference to specific embodiments thereof, it will be apparent to one of ordinary skill in the art that various changes and modifications can be made therein without departing from the spirit and scope thereof.

1-23. (canceled)

24. A compound of formula R1-R2-R3, wherein:
R1 is a hexacyclen;
R3 is a β-nicotinamide adenine dinucleotide; and
R2 is a moiety that links R1 and R3, wherein said moiety is a flexible linker.

25. The compound of claim 24, wherein R2 is a polyglycine moiety containing between 1 and 3 glycines.

26. The compound of claim 24, wherein R2 is selected from the group consisting of polyethylene glycol (PEG), polystyrene-PEG, a [2-(2-aminoethoxy)ethoxy] acetic acid, allyloxycarbonyl-[2-(2-aminoethoxy)ethoxy] acetic acid, fluorenyl-methoxycarbonyl-[2-(2-aminoethoxy)ethoxy] acetic acid, ter-butyloxycarbonyl-[2-(2-aminoethoxy)ethoxy] acetic acid, benzyloxycarbonyl-[2-(2-aminoethoxy)ethoxy] acetic acid, and BMPS (N-(β-maleimido-propyloxy)succinimide).

27. A compound of the formula R1-R2-R3, wherein:
R1 comprises a moiety that binds to a hemoglobin binding site on a cytochrome b₅ at one or more amino acids selected from the group consisting of H26, E43, E44, E48, A54, D60, H80 and A88, wherein said moiety is a polyamine;
R3 comprises a moiety that binds to the cytochrome b₅ at one or more amino acids selected from the group consisting of I24, L25, H26 and H27, wherein said moiety is ATP or an ATP analog; and
R2 comprises a moiety that links R1 and R3, wherein said moiety is a flexible linker.

28. The compound of claim 27, wherein the polyamine is linear or cyclic.

29. The compound of claim 27, wherein R1 is hexacyclen.

30. The compound of claim 27, wherein R3 is β-nicotinamide adenine dinucleotide.

31. The compound of claim 27, wherein R3 is 1,N6-ethenoadenosine 5′ triphosphate, β-nicotinamide adenine dinucleotide, 1,N6-ethenoadenosine hydrochloride, nicotinamide-1,6-ethenoadenosine, or coenzyme A.

32. The compound of claim 27, wherein R1 is hexacyclen and R3 is β-nicotinamide adenine dinucleotide.

33. The compound of claim 27, wherein R2 is a polyglycine moiety containing between 1 and 3 glycines.

34. The compound of claim 27, wherein R2 is selected from the group consisting of polyethylene glycol (PEG), polystyrene-PEG, [2-(2-aminoethoxy)ethoxy] acetic acid, allyloxycarbonyl-[2-(2-aminoethoxy)ethoxy] acetic acid, fluorenyl-methoxycarbonyl-[2-(2-aminoethoxy)ethoxy] acetic acid, ter-butyloxycarbonyl-[2-(2-aminoethoxy)ethoxy] acetic acid, benzyloxycarbonyl-[2-(2-aminoethoxy)ethoxy] acetic acid, and BMPS (N-(β-maleimido-propyloxy)succinimide).

35. A pharmaceutical composition comprising the compound of claim 24 or a pharmaceutically acceptable salt thereof.
